(19) United States
(12) Patent Application Publication
(10) Pub. No.: US 2008/0275133 A1
(43) Pub. Date: Nov. 6, 2008

(54) Local Administration of Retinoids to Treat Deficiencies in Dark Adaptation

(75) Inventors: Daniel M. Schwartz, San Francisco, CA (US); Mark E. Davis, Pasadena, CA (US)

Correspondence Address: FULBRIGHT & JAWORSKI, LLP, 1301 MCKINNEY, SUITE 5100, HOUSTON, TX, 77010-3095, US

(73) Assignee: University Of Calfornia-San Francisco, San Francisco, CA (US)

(21) Appl. No.: 11/576,377

(22) PCT Filed: Sep. 30, 2005
(86) PCT No.: PCT/US05/35294

§ 371 Date: Feb. 26, 2008

Related U.S. Application Data
(60) Provisional application No. 60/614,623, filed on Sep. 30, 2004.

(51) Int. Cl. A61K 31/07; A61P 27/02

(52) U.S. Cl. 514/725

(57) ABSTRACT

The present invention relates to improving, at least in part, a deficiency in dark adaptation for an individual. The therapy for dark adaptation includes local administration of a retinoid, such as a Vitamin A or a derivative thereof, such that deleterious side effects seen with systemic administration are avoided.

### CROSS-REFERENCE TO RELATED APPLICATIONS

The present invention claims priority to PCT Patent Application Serial No. PCT/US2005/035294, filed Sep. 30, 2005, and also to U.S. Provisional Patent Application Ser. No. 60/614,623, filed Sep. 30, 2004, both of which are incorporated by reference herein in their entirety.

### FIELD OF THE INVENTION

The present invention is generally directed to the fields of opthalmology, molecular biology, cell biology, chemistry, and medicine. Specifically, it relates to treatment of dark adaptation deficiency with local administration of retinoids, such as Vitamin A and/or its derivatives, or precursors to retinoids, such as beta carotene.

### BACKGROUND OF THE INVENTION

Elderly patients, especially those with age-related macular degeneration (AMD), often complain of visual difficulties at night. These consist of difficulty seeing in dim illumination, but more commonly, a marked slowing of dark adaptation. Dark adaptation is a measure of how rapidly the retina recovers sensitivity after a brief flash of light. This process is prolonged in elderly patients and is visually disabling. For example, many otherwise capable elderly people with abnormal dark adaptation cannot drive from daylight conditions into a tunnel and see in the darker environment; nor, for example, can they see well when they move from an illuminated lobby into a darkened movie theater. Currently, there is no suitable treatment for abnormal dark adaptation.

One group of patients with pronounced and early onset dark adaptation abnormalities are those with Sorsby's fundus dystrophy. In this condition, there is marked thickening of Bruch's membrane, which is an extracellular matrix situated between the retina and its blood supply, the choroid. Dark adaptation abnormalities rapidly normalize in these patients after receiving high dose oral vitamin A for as little as 1 week. While the reversal of the dark adaptation abnormality is dramatic in these patients, chronic high dose oral vitamin A is not a practical therapy because of associated systemic toxicity (liver damage, osteoporosis, promotion after receiving high dose oral vitamin A for as little as 1 week. While the reversal of the dark adaptation abnormality is dramatic in these patients, chronic high dose oral vitamin A is not a practical therapy because of associated systemic toxicity (liver damage, osteoporosis, promotion of lung cancer in smokers, etc.) Exemplary conditions associated with chronic toxicity may include at least some of the following: hypercalcemia; dry scaly skin; bone pain; changes in texture of hair and nails; increased cerebrospinal pressure; pruritus; headache; nausea; irreversible bone changes (e.g., demineralization); thinning of long bones; cortical hyperostosis; periostosis; and/or premature closing of epiphyses.

In aging, similar to Sorsby's fundus dystrophy, thickening of Bruch's membrane occurs. These changes in Bruch's membrane are accentuated in AMD. It is possible that high dose vitamin A supplementation might improve dark adaptation in these patients just as it does in patients with Sorsby's fundus dystrophy. However, because of the systemic toxicity associated with systemic administration, chronic high dose vitamin A therapy is not used in elderly patients to improve dark adaptation.

Thus, the present invention satisfies a long-felt need in the art for non-toxic therapies of dark adaptation deficiencies.

### SUMMARY OF THE INVENTION

The present invention concerns treatment for deficiencies in dark adaptation. In particular, the treatment regards local administration of one or more agents to partially or fully improve, ameliorate, reduce the intensity of, and/or prevent deficiencies in dark adaptation by local administration of a therapeutically effective amount of one or more agents. In specific embodiments, the one or more agents comprise one or more retinoids, such as Vitamin A or a derivative thereof; one or more precursors to a retinoid, such as a carotenoid (including beta carotene); or a mixture thereof. In further specific embodiments, the one or more agents are comprised in a pharmaceutically acceptable excipient.

In particular embodiments of the invention, one or more retinoids, such as all-trans retinol (Vitamin A), all-trans retinyl esters, 11-cis retinol, 11-cis retinal, or a combination thereof, are given in sufficiently high concentrations locally so that they could be absorbed by the RPE and/or retina such that dark adaptation abnormalities of individuals in need thereof, including the elderly, could be mitigated without associated systemic toxicity resulting from high dose oral vitamin A therapy. In a specific embodiment, the retinoids may be referred to as Vitamin A and its visual pigment derivatives. In further embodiments, the retinoids are visually active retinoids, which may be defined as retinoids that ameliorate at least one symptom of a dark adaptation abnormality or deficiency. In an analogous manner, a precursor to a retinoid is administered.

In particular embodiments, Vitamin A and/or its visual pigment derivatives can be administered locally in a suitable manner, such as by periocular injection, retrobulbar injection, or intraocular injection, for example. The agent(s) can be delivered in a sustained release formulation in any suitable method, such as by a periocular (sub-tenons injections), retrobulbar, or intraocular route, for example. The agent(s) could also be administered via trans-scleral or intraocular sustained release delivery devices, for example. In an alternative embodiment, the composition is not administered intraocularly.

In an embodiment of the present invention, there is a method of treating a deficiency in dark adaptation in an individual, comprising administering locally to at least one eye of the individual an effective amount of a retinoid, a precursor to a retinoid, or a mixture thereof. In specific embodiments, the retinoid is further defined as a visual cycle retinoid. In other specific embodiments, the retinoid or precursor to the retinoid are protected from light, oxygen, or both, and in particular aspects of the invention this is further defined as the retinoid or precursor to the retinoid being formulated in a composition with beta cyclodextrin, for example.

In particular embodiments, the individual has a thickened Bruch's membrane and/or the dark adaptation may be a result of a disease, the result of aging, or both. In a specific embodiment, the disease is macular degeneration, Sorsby's fundus dystrophy, retinitis pigmentosa, or idiopathic polypoidal vasculopathy, for example.

The administration of the retinoid and/or precursor may be in a sustained release composition, in specific aspects of the invention. In a specific embodiment, the local administration is further defined as administering the retinoid or precursor to the retinoid to a portion of the eye, said portion comprising Bruch's membrane, the sclera, the retina, the retinal pigment epithelium, the choroid, the macula, the vitreous, the anterior/posterior chamber and/or in the subretinal space. The local administration may be by periocular administration, retrobulbar administration, intraocular administration, or a combination thereof. In another specific embodiment, the local administration is by injection or topically. The retinoid and/or the precursor to the retinoid may be comprised in a sustained release configuration.

In a specific embodiment of the invention, the retinoid is further defined as Vitamin A, a derivative of Vitamin A, or a mixture thereof. In a further specific embodiment, a derivative of Vitamin A is 11-cis retinal, 11-cis retinol, an all-trans retinyl ester, an all-trans retinal, or a mixture thereof. In a particular aspect, the precursor to the retinoid comprises a carotenoid, such as beta carotene, for example.

### BRIEF DESCRIPTION OF THE DRAWINGS

FIG. 1 illustrates a schematic of the reactions of the visual cycle involved in the interconversion of vitamin A and 11-cis retinal (from Thompson and Gal, 2003).

FIG. 2 illustrates exemplary retinoids in the invention (from the IUPAC-IUB Joint Commission on Biochemical Nomenclature (JCBN), world wide web version, as reported by G. P. Moss (Arch. Biochem. Biophys., 1983 224, 728-731; Eur. J. Biochem., 1982, 129, 1-5; J. Biol. Chem., 1983, 258,5329-5333; Pure Appl. Chem., 1983, 55, 721-726; Biochemical Nomenclature and Related Documents, 2^{nd} edition, Portland Press, 1992, pages 247-251)).

FIG. 3 demonstrates an exemplary dark adaptation curve (Owsley et al., 2001).

### DESCRIPTION OF ILLUSTRATIVE EMBODIMENTS

It will be readily apparent to one skilled in the art that various substitutions and modifications may be made in the invention disclosed herein without departing from the scope and spirit of the invention.

### I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. In certain aspects, one or more compositions and/or methods of the invention may consist of or consist essentially of one or more embodiments. Also, one of skill in the art recognizes that a particular embodiment of the invention is exemplary in nature and will apply to other embodiments of the invention.

The term “age-related macular degeneration (AMD)” as used herein is referred to as macular degeneration in an individual over a particular age, such as the age of about 50. In one specific embodiment, it is associated with Drusen and/or thickening of Bruch's membrane. In a particular embodiment of the invention, dark adaptation is one symptom of AMD. In specific embodiments, other degenerations are included in the scope of the term, such as Sorsby's fundus dystrophy.

The term “Bruch's membrane” as used herein refers to a five-layered structure separating the choriocapillaris from the RPE.

The ten-n “dark adaptation” as used herein refers to the measure of how rapidly the retina recovers sensitivity after a brief flash of light or sustained light exposure.

The term “deficiencies in dark adaptation” refers to any abnormality associated with the ability of adapting to darkened environments, such as a marked slowing of dark adaptation, difficulty in seeing in dim illumination (scotopic sensitivity), or both, for example. In a specific embodiment, it refers to an abnormality in the increase in visual sensitivity with increasing time in the dark following exposure to light.

The term “macula” as used herein refers to the central area of the retina, including light-sensing cells of the central region of the retina.

The term “macular degeneration” as used herein refers to deterioration of the central area of the retina, the macula.

The term “precursor to a retinoid” as used herein refers to a compound that indirectly or directly is metabolized into a retinoid. In a specific embodiment, the precursor is a carotenoid. In another embodiment, the precursor is beta carotene. In particular, an enzyme required to convert the precursor to the retinoid is present in the tissue to which the precursor is delivered (see, for example, Bhatti et al., 2004), which may be the retina, RPE, choroid, or combination thereof, for example, although in alternative embodiments the enzyme is delivered concomitantly with the precursor.

As noted in the art (see, for example, Stahl et al. (2005)), carotenoids may be considered to be provitamin A and non-provitamin A compounds. Although α-carotene, and β-cryptoxanthin contribute to vitamin A supply and may prevent vitamin A deficiency, beta-carotene is the major provitamin A carotenoid in the Western diet. The promotion of growth, embryonal development and visual function requires Vitamin A. Daily vitamin A intake in the form of provitamin A carotenoids depends on dietary habits and available food sources, and in Western societies the intake of carotenoids is far less than the 70% of the vitamin A intake in ingested from fruits and vegetables in Third World countries. However, intake requires adjustment for bioequivalence, such as by evaluating what portion of the ingested provitamin A carotenoid is absorbed, cleaved, reduced and ultimately available as retinol or retinyl ester. Such conditions as food matrix properties, preparation of the food, coingestion of fat and fiber, diseases of the gastrointestinal tract, vitamin A status and/or malnutrition can affect bioavailability and metabolism. There is evidence that a variety of enzymes targeted to different cleavage sites metabolize carotenoids to apo-carotenoids and retinal. Enzymes having β,β-carotene 15,15′-oxygenase activity have been cloned from multiple organisms, including human, mouse, Drosophila, and chicken. A variety of factors can determine vitamin A supply via carotenoids, including, for example, the efficacy of cleavage, substrate specificity for various provitamin A compounds, and multiple genetic variations and factors that impact on the expression of carotenoid-metabolizing enzymes.

The term “retina” as used herein refers to the neurological tissue at the posterior of the eye, containing the rods and cones that receive light and convert it to electrical signals for transmission via the optic nerve to the brain.

The term “retinal pigment epithelium” or “RPE” refers to the cuboidal epithelial monolayer that is situated between the neural retina and choroid. The RPE derives developmentally from, and is contiguous with, the same neuroectodermal layer as the neural retina. The RPE possesses numerous large pigment granules (melanosomes) that participate in the prevention of light scattering. In addition, the RPE plays a critical role in the maintenance of photoreceptor cell viability and function by the phagocytosis and removal of photoreceptor outer segment disks, the processing and secretion of various molecules necessary for photoreceptor function and viability (such as vitamin A derivatives and growth factors), the regulation of macromolecular traffic between the retina and choroid, and the mediation of retinal adhesion.

The term “retinoid” as used herein refers to a class of compounds comprising four isoprenoid units joined in a head-to-tail manner. In specific embodiments, all retinoids may be formally derived from a monocyclic parent compound comprising five carbon-carbon double bonds and a functional group at the terminus of the acyclic segment. In a specific embodiment, the retinoid is a visually active retinoid, such as one capable of ameliorating at least one symptom of a dark adaptation deficiency. In particular embodiments, the retinoid facilitates absorption of the administered retinoid or a derivative thereof by the RPE and/or the retina. In certain aspects of the invention, the retinoid is further defined as a visual cycle retinoid, which refers to those retinoids in the visual cycling of vitamin A derivatives between the retina and the retinal pigment epithelium (RPE).

The term “therapeutically effective” as used herein refers to the amount of a composition able to partially or fully improve, ameliorate, reduce the intensity of and/or prevent at least one symptom of dark adaptation.

### II. The Present Invention

The invention generally concerns treatment of an individual's insufficient ability to adapt ocularly to the dark. Individuals with this abnormal dark adaptation, which may be referred to as impaired dark adaptation or night blindness, see substantially normally when adequate amounts of light are present, but see poorly in the darkness, and/or upon going from a well-lit to a dimly-lit environment. Symptoms include difficulty seeing when driving in darkened conditions, such as at night or in a tunnel, glare from lights while driving at night, poor vision in reduced light conditions, and/or the sensation that the eyes take longer to “adjust” to seeing in the dark, for example.

The cellular processes associated with impaired dark adaptation are generally known and involve the retina, located at the back of the eye, which senses light upon passing through the lens and converts the light to a nerve impulse for interpretation by the brain. This process is as follows. Retinol, which is present in the circulation, is transported to the retina where it moves into retinal pigment epithelial cells. There, retinol is esterified to form a retinyl ester, and, when needed, the esters are hydrolyzed and isomerized to form 11-cis retinal. The 11-cis retinal can be shuttled across the interphotoreceptor matrix to the rod outer segment, where it binds to the opsin protein to form the visual pigment, rhodopsin.

Rod cells with rhodopsin can detect very small amounts of light, making them important for night vision. That is, absorption of a photon of light catalyzes the isomerization of 11-cis retinal to all-trans retinal. This isomerization ultimately leads to the generation of an electrical signal to the optic nerve, which is conveyed to the brain for interpretation as vision. Once released, all-trans retinal is converted to all-trans retinol, which can be transported across the interphotoreceptor matrix to the retinal epithelial cell to complete the visual cycle. Inadequate 11-cis-retinal availability to the retina results in impaired dark adaptation. In particular embodiments of the present invention, a thickened Bruch's membrane is responsible at least in part for such inadequacy. In other embodiments, the dark adaptation deficiency is due to inadequate regeneration of 11-cis retinal by the RPE, or by impaired transport of retinoids from the RPE to the retina and vice versa.

### III. Dark Adaptation

In dark adaptation, the process of adjusting the eyes to low levels of illumination occurs. Cones may adapt first, followed by rods continuing to adapt for up to about forty minutes, in certain aspects. Moreover, during dark adaptation the sensitivity of the eye to light is increased.

The eye possesses a remarkable capability to become sensitive to light. Light perception of the eye can increase by a factor of 10 billion from full sunlight to the least light perceptible. Two major components of dark adaptation include dilatation of the pupil and the photochemical alterations of the retina.

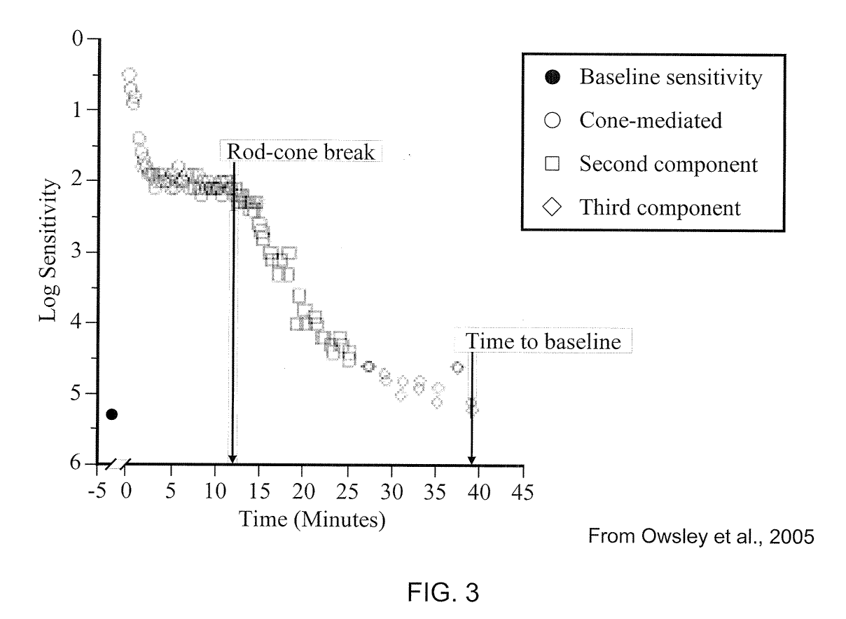

In a typical investigation of dark adaptation, an observer is exposed to a bright light flash. The adaptation stimulus is extinguished, and thresholds are measured for dim light stimuli while the observer sits in complete darkness. The intensity of the just-detectable light stimulus and the time at which it was detected are recorded, and the process repeats itself over and over for approximately 30 minutes, in some embodiments. Initially, the threshold is quite high, but it gradually declines and appears to reach a lower asymptote after approximately 6 minutes, in specific embodiments. However, at around 7 minutes, for example, thresholds begin to decline again, and reach a second (much lower) asymptote after approximately 30 minutes, in particular embodiments. An exemplary dark adaptation curve from Owsley et al. (2001) is provided in FIG. 3.

The time course of dark adaptation is well-known. The function is bipartite: one for the cone receptors and the other for the rods. The receptors contain photopigments in their outer segments, and upon absorption of light by these photopigments, particular changes prevent them from helping to send visual signals to the brain; these changes are reversed in darkness.

The normal visual system of a human is most sensitive when the photopigments have not absorbed any light for about 30 minutes. Under these conditions, the photopigments are considered to be fully regenerated. When the rod photopigments, referred to as rhodopsin, are exposed to light, they undergo a process referred to as bleaching, because the photopigment color actually become almost transparent. In the dark the photopigments regenerate and regain their pigmentation.

In a review by Lamb and Pugh (2001), a classic result from the dark adaptation literature is provided in a figure reproduced from Hecht et al. (1937). As stated by Lamb and Pugh, the vertical axis on a logarithmic scale is the threshold intensity required for an observer to detect a visual stimulus, which is plotted against time after the extinction of a bleaching exposure; this is performed for five different levels of bleach. After the exposure having the greatest intensity, which produced a near-total bleach of pigment, the observer's visual threshold was elevated by at least 5 orders of magnitude initially and then recovered with a characteristic biphasic form. Lamb and Pugh then state: “Over the first several minutes there was rapid recovery to a plateau level 3 log units above the dark-adapted value, and this rapid phase is known to be mediated by the cone photoreceptor system. After about 11 min, the threshold began dropping again as the rod system became more sensitive than the cone system, and recovery proceeded steadily at first but then later more slowly, so that it took more than 40 min to attain the original fully dark-adapted sensitivity.”

The cone receptors (most of which are in the fovea) also have outer segments that contain photopigments. The photopigments in the cones also bleach when exposed to light. They are divided into three classes of cone photopigments, each class of which is photochemically a little different than the other, and therefore their spectral absorbencies are different.

In some embodiments, for approximately the first 10 minutes in the dark, the cones require less light to reach a threshold response than do the rods. Thereafter, the rods require less light. The point at which the rods become more sensitive is called the rod-cone break.

In the present invention, one or more of the above-mentioned dark adaptation processes may be deficient, non-functioning, impaired, damaged, and/or weakened. The dark adaptation deficiency of the present invention may be the result of normal aging process(es), disease, or both. In particular, deficiencies in dark adaptation are often the result of thickening of Bruch's membrane caused by any process or disease; normal aging; macular degeneration, such as age-related macular degeneration; Sorsby's fundus dystrophy; idiopathic polypoidal vasculopathy; fundus albipunctatus; Bothnia dystrophy; Stargardt macular dystrophy; and/or normal aging processes. In particular, the dark adaptation deficiency is treated locally in the eye, which may also be referred to as in a non-systemic manner. In a specific embodiment, the individual being treated for dark adaptation has an ocular Vitamin A deficiency, and although the individual may be treated with Vitamin A or a derivative thereof in a systemic manner, the therapy for the ocular deficiency is local in manner due to toxicities with prolonged systemic treatment with Vitamin A or its derivatives. A dark adaptation abnormality may include a marked slowing of dark adaptation, difficulty in seeing in dim illumination, or both, for example.

### IV. Tests for Abnormal Dark Adaptation

In particular, an individual may be tested for having a normal range of dark adaptation. In particular, the individual may be suspected of having abnormal dark adaptation ability, such as an individual over about the age of 50, an individual with a thickened Bruch's membrane, an individual with an ocular disease, such as macular degeneration, including age-related macular degeneration, Sorby's fundus dystrophy, and/or combinations thereof.

Although multiple methods of testing dark adaptation are available, in a particular embodiment the individual is tested according to Jackson et al. (1999), for example. After baseline sensitivity measurement, a test eye undergoes a bleach (0.25 ms) using an electronic flash of white light (Sunpak 622 Super, Tocad, Ltd.) that produced a measured intensity of 7.65 log scotopic Trolands, which produces an expected ˜98% bleach in the affected area of the retina to be tested (Pugh, 1975). Threshold measurements begin immediately subsequent to flash offset, and an external microcomputer Macintosh 840AV, Apple, Inc.) controls the psychophysical procedure and recorded responses. To estimate threshold, a three down-one up modified staircase threshold procedure was used with a target intensity starting at 4.85 cd/m². Targets were presented every 2-3 s for a duration of 200 ms, and the subject's eye with the better acuity was tested. However, if the acuity was the same in both eyes, the right eye was tested.

The subject's test eye is aligned to the fixation light using the camera built into the HEA with a distance from the subject's test eye to the fixation light being about 30 cm. The subject presses a response button when the target is visible, although only 750 ms are given to make a response after target arrival. If no response to the target is made, the target intensity remains at 4.85 cd/m² until the subject responds. If the subject indicates the target is visible, the target intensity is decreased by 0.3 log units steps in succession until the subject stops responding that the target is present. After the subject responds that the stimulus is invisible, target intensity is increased by 0.1 log units until the subject responds that the target is once again visible. This target intensity is defined as threshold. Successive threshold measurements start with a target intensity 0.3 log units brighter than the previous threshold estimate, and threshold estimates are made twice every minute for the first 25 min and twice every 2 min thereafter. Dark adaptation measurement stops when the sensitivity of the subject is within 0.3 log units of the previously measured baseline sensitivity. To control for pupil size, subjects are dilated with 1% tropicamide and 2.5% phenylephrine hydrochloride prior to testing.

Improvements in dark adaptation deficiencies may be assessed following treatment with the present invention using the aforementioned methods or others known or otherwise suitable in the art.

### V. Retinoids

Retinoids generally describe a large number of related compounds, such as Vitamin A (a generic term) and derivatives of Vitamin A. In certain aspects of the invention, the retinoid is further defined as a visual cycle retinoid, which refers to those retinoids in the visual cycling of vitamin A derivatives between the retina and the retinal pigment epithelium (RPE).

Exemplary retinoids include retinol, retinal, retinoic acid, retinaldehyde, and others. Retinoids include both naturally occurring compounds with vitamin A activity and synthetic analogs of retinoic acid. In particular embodiments, 11-cis retinal is utilized. Beta-carotene may also be employed, given that β,β-carotene-15,15′-oxygenase is present in the RPE.

The active retinoids occur in 3 forms: alcohol (retinol), aldehyde (retinal or retinaldehyde), and acid (retinoic acid). Inactive retinoids, known as provitamins A, are produced as plant pigments and are called carotenoids. Several hundred carotenoids occur in foods, but only approximately 50 can be metabolized into the active retinoid forms; among these 50 compounds, beta-carotene, a retinol dimer, has the most significant provitamin A activity.

In the human body, retinol is the predominant form, and 11-cis-retinol is the active form. Retinol-binding protein (RBP) binds vitamin A and regulates its absorption and metabolism.

Vitamin A (retinol) is a fat-soluble vitamin found mainly in fish liver oils, liver, egg yolk, butter, and cream. Green leafy and yellow vegetables contain beta-carotene and other provitamin carotenoids which are converted to retinol in the mucosal cells of the small intestine and also in peripheral tissues. Retinol cannot be synthesized in vivo and must be obtained from the diet. Retinol is metabolized into the biologically active derivative retinoic acid (RA) in a variety of cells. The 11-cis isomer of retinal (vitamin A₁ aldehyde), combined with a protein moiety, opsin, forms the prosthetic group of photoreceptor pigments in the retina that are involved in night, day, and color vision.

Toxic side-effects associated with retinoid treatments include changes in the skin and mucous membranes (dry skin, hair loss, dry nose, conjunctivitis), musculoskeletal symptoms, liver function abnormalities, osteoporosis, changes in clinical chemistry markers (increase in serum triglycerides and decrease in high-density lipoproteins) and, rarely, central nervous system effects.

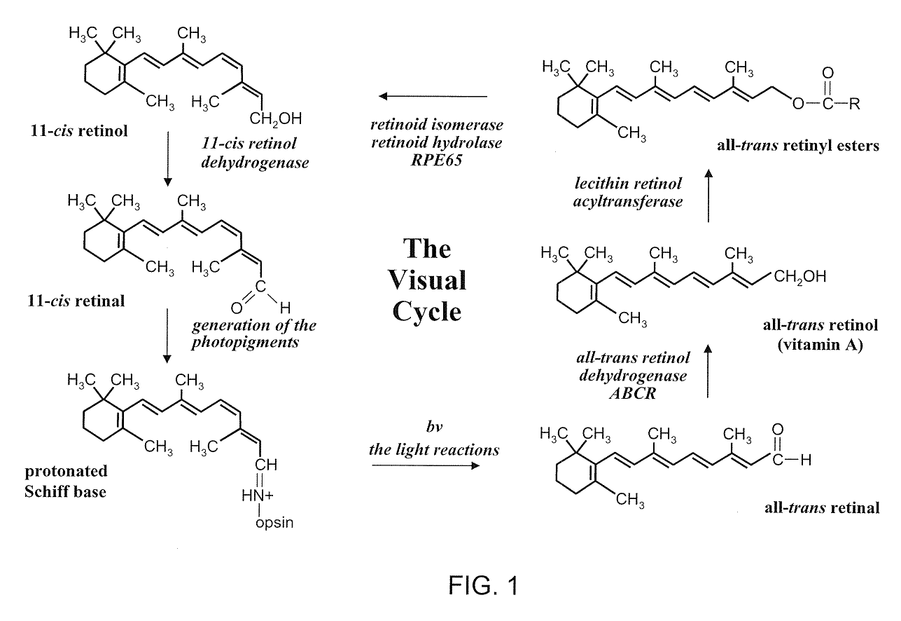

Retinoids are part of the visual cycle, a series of reactions presented in FIG. 1. In the normal visual cycle, vitamin A (all-trans retinol) circulates bound to retinol binding protein and transthyretin. It is taken up by the retinal pigment epithelium (RPE) and converted to its active form 11-cis retinal by a series of enzymatic reactions. 11-cis retinal is then transported to the photoreceptor outer segments where it binds to opsin and forms the active retinal pigment, rhodopsin. Following absorption of light, 11-cis retinal is isomerized to all-trans retinal. After all-trans retinal is “flipped” to the outer segment cytoplasm by ABCR, it is reconverted to all-trans retinol and then transported back to the RPE for reconversion to 11-cis retinal.

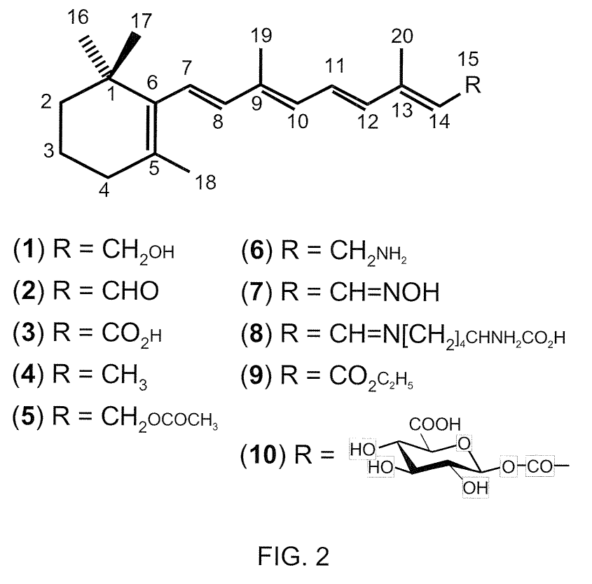

In specific embodiments, the term “Vitamin A” is generally used for retinoids exhibiting qualitatively the biological activity of retinol. FIG. 2 illustrates exemplary retinoids that may be employed in the invention. As shown therein, the compound (1) denotes (2E,4E,6E,8E)-3,7-dimethyl-9-(2,6,-trimethylcyclohex-1-en-1-yl)nona-2,4,6,8-tetraen-1-ol, which is also referred to as vitamin A, vitamin A alchohol, vitamin A₁, vitamin A₁ alchohol, axerophthol or axerol, and retinol. Compound (2) refers to vitamin A aldehyde, vitamin A₁ aldehyde, retinene or retinenl, retinal, or retinaldehyde. Compound (3) represents tretinoin, vitamin A acid, vitamin A₁ acid, or retinoic acid. Compound (4) refers to axerophthene or deoxyretinol. Exemplary derivatives of the basic hydrocarbon include exemplary compound (5), which is retinyl acetate, and exemplary compound (6), which is retinylamine. Exemplary derivatives of retinal include retinal oxime (compound (7)) and N⁶-retinylidene-L-lysine (compound (8)). Exemplary derivatives of retinoic acid include ethyl retinoate (compound (9)) and 1-O-retinoyl-□-D-glucopyranuronic acid (compound (10)). Beta-carotene may also be employed in the invention.

In particular embodiments of the invention, 11-cis retinal, 11-cis retinol, an all-trans retinyl ester, an all-trans retinal, beta-carotene, or a mixture thereof are administered to an individual in need thereof for improvement of dark adaptation abnormality. In a particular aspect of the invention, there is modification to the employed composition to stabilize (for example, increase t_{1/2}) against heat, photodegradation, or a combination thereof.

### VI. Formulations, Administration and Dosage

The compositions of the present invention are formulated so as to provide an effective concentration in the desired tissue. The term retinoids refers to synthetic retinoids, naturally-occurring retinoids, derivatives of either synthetic or naturally-occurring retinoids, or mixtures thereof. In particular embodiments, the retinoids are chemically modified to enhance their effectiveness, such as being formulated with or otherwise modified to include one or more compounds to protect the retinoid or precursor from at least oxygen and/or light. In a specific embodiment, the composition is utilized as a sustained released composition (see, for example, Example 3).

Any one or more pharmaceutically acceptable excipients appropriate to the particular retinoid may be used. Thus, the compound may be administered as an aqueous composition, as a topical composition, as a transmucosal or transdermal composition, in a local injection (such as periocular or intraocular) or a combination thereof. The formulation may also include liposomes, fibrin sealant, balanced salt solution, or a combination thereof.

The compositions of the present invention can be administered in any of a variety of ways, although in a particular embodiment the administration is local. Local administration of Vitamin A and/or a derivative thereof is preferably such that the localization facilitates absorption by the RPE and/or retina, thereby at least improving dark adaptation abnormalities. The compound may be placed directly in the eye, such as topically, for example in an eye drop or wash, or it may be injected around and/or into the eye, such as by periocular injection or intraocular injection, for example. The composition may be formulated in a sustained release composition for intraocular or periocular delivery. In some embodiments, the composition is delivered intraocularly, whereas in other embodiments the composition is not delivered intraocularly.

The dose of a retinoid is a suitable enough amount for improvement of at least one symptom of dark adaptation. As is generally recognized, there is a nexus between the retinoid, the formulation, the mode of administration, and the dosage level. Adjustment of these parameters to fit a particular combination is possible and routine.

### VII. Local Delivery of Compositions

In particular embodiments of the present invention, the retinoid composition is administered locally to treat dark adaptation deficiency. Although any suitable method for delivering the compounds such that at least one symptom of abnormal dark adaptation is treated, in particular embodiments the delivery is in a sustained release composition and/or configuration. Nevertheless, more than one administration of the retinoid may be required. In specific embodiments, there is a transcleral drug delivery, such as using a device to elute the drug in a periodic manner; in a sustained low dose manner; by periocular injection; by retrobulbar injection; or by intraocular injection. The agent may be delivered in a sustained release formulation, again by a periocular (sub-tenons injections), retrobulbar, or intraocular route.

For example, a method of local administration of the compositions of the invention may be employed such as one described in Ambati et al. (2000), wherein an osmotic pump was used for delivery of a compound across the sclera. In other embodiments, coulomb-controlled iontophoresis is utilized for delivery, such as is described in Behar-Cohen et al. (1997) wherein coulomb controlled iontophoresis allowed administration of dexamethasone for a therapeutic effect on the posterior as well as the anterior segment of the eye.

Another example of ocular drug delivery is described in U.S. Pat. No. 5,466,233, which is incorporated by reference herein in its entirety. As described therein, there is a tack comprising a post, an anchoring region and a head. The post is for being positioned within the vitreous region of the eye. The post has a first end and a second end, and includes a drug to be administered. The anchoring region is affixed to the second end of the post, and includes a width measured perpendicularly to a longitudinal axis of the tack, which varies to provide the anchoring region with a configuration to anchor the tack within at least one of a sclera, a retina and a choroid. The head extends radially outwardly from the anchoring region such that upon insertion of the anchoring region and post within the eye, the head remains external to the eye and abuts a scleral surface of the eye.

U.S. Pat. No. 6,375,972, which is incorporated by reference herein in its entirety, describes a sustained release drug delivery system comprising an inner drug core having a drug; an inner tube impermeable to the passage of the drug, wherein the inner tube includes first and second ends and covers at least a portion of the inner drug core, wherein the inner tube is sized and formed of a material so that the inner tube is dimensionally stable to accept the drug core without changing shape. An impermeable member is positioned at the inner tube first end, and the impermeable member prevents passage of the agent out of the drug core through the inner tube first end, and a permeable member is positioned at the inner tube second end, wherein the permeable member allows diffusion of the drug out of the drug core through the inner tube second end. The delivery system may be applied to the vitreous of the eye, under the retina, or onto the sclera, for example.

Another exemplary delivery device for the retinoids of the invention include that of U.S. Pat. No. 6,413,540, which is incorporated by reference herein in its entirety, wherein there is an ophthalmic drug delivery device, comprising a body having a scleral surface for placement proximate a sclera and a well having an opening to the scleral surface, wherein there is an inner core disposed in the well comprising the drug.

Also, U.S. Pat. No. 6,756,049, which is incorporated by reference herein in its entirety, regards a sustained release drug delivery device comprising a) a drug core comprising a drug for a desired local effect; b) a unitary cup essentially impermeable to the passage of the drug that surrounds and defines an internal compartment to accept the drug core, the unitary cup comprising an open top end with at least one recessed groove around at least some portion of the open top end of the unitary cup wherein the unitary cup further comprises an integral suture tab; and c) a permeable plug which is permeable to the passage of the drug, the permeable plug is positioned at the open top end of the unitary cup wherein the groove interacts with the permeable plug holding it in position and closing the open top end, the permeable plug allowing passage of the agent out of the drug core, through the penneable plug, and out the open top end of the unitary cup.

In particular embodiments, a composition of the invention can cross the sclera and choroid to the vitreous where it can then diffuse to target tissue (such as, for example, the retina or RPE); it can also traverse the scleral, choroid and Bruch's membrane to reach the RPE and/or retina. In other embodiments, the compositions may be given intraocularly (into the vitreous, anterior/posterior chamber or in the subretinal space, for example).

In additional embodiments, drug encapsulation systems are utilized that provided sustained release, including the following: liposomes, biodegradable microspheres, cylinders, and capsules, such as those made of lactic and glycolic acid and hydrogels. In further embodiments, membrane-enclosed reservoir devices, monolithic systems, and nano-particles may be employed.

### EXAMPLES

The following examples are offered by way of example, and are not intended to limit the scope of the invention in any manner.

### Example 1

### Exemplary Embodiment of Retinoid Administration

A 70 year-old patient complains of difficulty driving at night, especially in going from a well-lit to a poorly-illuminated environment. Dark adaptation is tested and is abnormally prolonged. One cc of Vitamin A and/or one of its visual pigment derivatives in a sustained release suspension is injected retrobulbarly on both sides. One week later, the patient drives without difficulty at night. Six months later, the retrobulbar injections are repeated.

Any of the exemplary agents depicted in FIG. 1 as a component of the visual cycle or in FIG. 2, for example, could be administered alone or in combination with other components.

### Example 2

### Additional Exemplary Embodiment of Retinoid Administration

A 65 year-old patient complains of difficulty driving at night, especially in going from a well-lit to a poorly-illuminated environment. Dark adaptation is tested and is abnormally prolonged. The patient has a cataract in the right eye and cataract surgery with an intraocular lens is performed. At the time of surgery, a small pellet of concentrated vitamin A and/or one of its visual pigment derivatives is placed in the eye for sustained release. One week later, the patient drives without difficulty at night. Vitamin A and/or one of its visual pigment derivatives is delivered in effective concentrations to the retina over the next 0.5-3 years.

### Example 3

### Shielding and/or Photostability Increase of Composition

Several issues may arise when using a sustained release of vitamin A in the eye. Vitamin A and its esters are well-known to be sensitive to oxygen and to light. Thus, it is advantageous to employ slow-release methodologies that would protect the vitamin A from light- and oxygen-assisted degradation while in the sustained release formulation and/or device. Beta cyclodextrin (BCD), for example, is known to form inclusion complexes with vitamin A and related compounds. Of the numerous papers demonstrating inclusion complex behavior of vitamin A and its related compounds and BCD, several are of direct importance to this application. Two concepts useful to the present invention are the following: (i) it has been shown that BCD can shield retinal from oxygen by forming an inclusion complex (Lerner et al., 1989); and (ii) it has been demonstrated that BCD can increase the photostability of all-trans-retinoic acid by inclusion complex formation (Lin et al., 2000). Thus, specific embodiments of sustained vitamin A delivery in the eye would involve devices that comprise BCD to protect the vitamin A against degradation via oxygen and light. As an example of one device, described in U.S. patent application Ser. No. 11/148,011, filed on Jun. 7, 2005, which is incorporated by reference herein in its entirety, there are wet spinning methods to produce sutures that contain inclusion complexes of drugs and BCD that are contained within the body of the fiber. The polymeric fibers are degradable over time. These sutures containing antibiotics have been prepared and implanted in the eyes of rabbits. Drug released into the eye has been monitored from these sutures and neither the released drug nor the suture produced local inflammation or any other indications of toxicity. In a manner similar to the antibiotic, vitamin A or any related retinoid or precursor thereto can form inclusion complexes with BCD, for example, and be incorporated into the sustained release fiber by wet spinning.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference herein.

### PATENTS

U.S. Pat. No. 6,375,972
U.S. Pat. No. 6,413,540
U.S. Pat. No. 6,756,049

### PUBLICATIONS

Ambati, J., Gragoudas, E. S., Miller, J. W., You, T. T., Miyamoto, K., Delori, F. C., and Adamis, A. P. Transscleral delivery of bioactive protein to the choroid and retina. Invest. Opthalmol. Vis. Sci. 41:1186-1191, 2000.
Behar-Cohen F F, Parel J M, Pouliquen Y, Thillaye-Goldenberg B, Goureau O, Heydolph S, Courtois Y, De Kozak Y. Iontophoresis of dexamethasone in the treatment of endotoxin-induced-uveitis in rats, Exp Eye Res. 65(4):533-45, 1997.
Bhatti R A, Yu S, Boulanger A, Fariss R N, Guo Y, Bernstein S L, Gentleman S, Redmond T M., Expression of beta-carotene 15,15′ monooxygenase in retina and RPE-choroid., Invest Opthalmol V is Sci. 44(1):44-9, 2003.
Hecht, S., Haig, C., and Chase, A. M. The influence of light adaptation on subsequent dark adaptation of the eye; J. Gen. Physiol., 20:831-850, 1937.
Lamb T D, Pugh E N Jr. Dark adaptation and the retinoid cycle of vision. Prog Retin Eye Res., 23(3):307-80, 2004.
Lerner, D. A., B. Del Castillo and S. Munoz-Botella, “Room Temperature Luminescence of a Retinal Complex of Cyclodextrin,” Analytica Chimica Acta, 227 (1989) 297-301.
Lin, H. S., C. S. Chean, Y. Y. Ng, S. Y. Chan and P. C. Ho, “2-Hydroxypropyl-beta-cyclodextrin increases aqueous solubility and photostability of all-trans-retinoic acid,” J. Clin. Pharmacy and Therapeutics, 25 (2000) 265-269.
Stahl W, Sies H. Biochimica et Biophysica Acta (BBA)—Molecular Basis of Disease Volume 1740, Issue 2, 30 May 2005, Pages 101-107.
Thompson D A, Gal A. Vitamin A metabolism in the retinal pigment epithelium:genes, mutations, and diseases. Progress in Retinal and Eye Research 22:683-703, 2003.
Vinton N E, Russell R M. Evaluation of a rapid test of dark adaptation. Am J Clin Nutr. 1981 September; 34(9): 1961-6.
Wright, K., B. Mack and M. E. Davis, “Biodegradable Drug-Polymer Delivery Systems,” US patent Appl (2005).

What is claimed is:

1. A method of treating a deficiency in dark adaptation in an individual, comprising administering locally to at least one eye of the individual an effective amount of a retinoid, a precursor to a retinoid, or a mixture thereof.

2. The method of claim 1, wherein the retinoid is further defined as a visual cycle retinoid.

3. The method of claim 1, wherein the retinoid and/or precursor to the retinoid are protected from light, oxygen, or both.

4. The method of claim 4, further defined as the retinoid and/or precursor to the retinoid being formulated in a composition with beta cyclodextnin.

5. The method of claim 1, wherein the administering of the retinoid and/or precursor to the retinoid is in a sustained release composition.

6. The method of claim 1, wherein the individual has a thickened Bruch's membrane.

7. The method of claim 1, wherein the dark adaptation is a result of a disease, is the result of aging, or both.

8. The method of claim 7, wherein the disease is macular degeneration, Sorsby's fundus dystrophy, retinitis pigmentosa, or idiopathic polypoidal vasculopathy.

9. The method of claim 1, wherein the local administration is further defined as administering the retinoid to a portion of the eye, said portion comprising Bruch's membrane, the sclera, the retina, the retinal pigment epithelium, the choroid, the macula, the vitreous, the anterior/posterior chamber and/or in the subretinal space.

10. The method of claim 1, wherein the local administration is by periocular administration, retrobulbar administration, intraocular administration, or a combination thereof.

11. The method of claim 1, wherein the local administration is by injection or topically.

12. The method of claim 1, wherein the retinoid is comprised in a sustained release configuration.

13. The method of claim 1, wherein the retinoid is further defined as Vitamin A, a derivative of Vitamin A, or a mixture thereof.

14. The method of claim 13, wherein a derivative of Vitamin A is 11-cis retinal, 11-cis retinol, an all-trans retinyl ester, an all-trans retinal, or a mixture thereof.

15. The method of claim 1, wherein the precursor to the retinoid comprises a carotenoid.

16. The method of claim 15, wherein the carotenoid is beta carotene.
